(19) United States
(12) Patent Application Publication
(10) Pub. No.: US 2007/0191327 A1
(43) Pub. Date: Aug. 16, 2007

(54) STERILIZATION OF CORTICOSTEROIDS WITH REDUCED MASS LOSS

(75) Inventors: Malcolm Hill, Solana Beach, CA (US); Cynthia LiCalsi, San Diego, CA (US)

Correspondence Address: WILSON SONSINI GOODRICH & ROSATI, 650 PAGE MILL ROAD, PALO ALTO, CA, 94304-1050, US

(73) Assignee: VERUS PHARMACEUTICALS, INC., San Diego, CA (US)

(21) Appl. No.: 11/675,563

(22) Filed: Feb. 15, 2007

Related U.S. Application Data
(60) Provisional application No. 60/774,152, filed on Feb. 15, 2006.
(60) Provisional application No. 60/774,073, filed on Feb. 15, 2006.
(60) Provisional application No. 60/774,151, filed on Feb. 15, 2006.

(51) Int. Cl. A61L 2/18; A61K 31/56

(52) U.S. Cl. 514/179; 422/28

(57) ABSTRACT

Novel methods of sterilizing corticosteroid solutions resulting in improved final yield of active corticosteroid ingredient.

### PRIORITY CLAIM AND CROSS-REFERENCE TO RELATED APPLICATIONS

This application claims the benefit of and priority under 35 U.S.C. §119(e) from United States Provisional Patent Application No. 60/774,152, filed on Feb. 15, 2006, which is incorporated herein by reference in its entirety. This application further claims the benefit of and priority under 35 U.S.C. 19(e) to U.S. provisional patent application 60/774,073, filed on Feb. 15, 2006, which is incorporated herein by reference in its entirety. This application further claims the benefit of and priority under 35 U.S.C. §119(e) from U.S. Provisional Patent Application No. 60/774,151, which was filed on Feb. 15, 2006, and which is incorporated herein by reference in its entirety.

This application is related to copending application Ser. No. 11/675,569, filed Feb. 15, 2007, entitled “Methods of Manufacturing Corticosteroid Solutions,” Attorney Docket Number 31622-718/201, which is incorporated herein by reference in its entirety. This application is also related to copending application Ser. No. 11/675,575, filed Feb. 15, 2007, entitled “Stable Corticosteroid Mixtures,” Attorney Docket Number 31622-719/201, which is incorporated herein by reference in its entirety.

### BACKGROUND OF THE INVENTION

Aqueous suspensions of budesonide are known. To date it has not been possible to sterilize such suspensions by filtration, as the micronized budesonide particles would clog the filter membrane, leading to excessive retention of the budesonide in and behind the filter membrane. Thus, however other methods of sterilization have proven undesirable for a variety of reasons including the complexity of sterilizing corticosteroid and the poor stability of the corticosteroid under such conditions.

Aqueous solutions of budesonide have been reported. See, for example, WO 2005/065649, WO 2005/065435 and WO 2005/065651 teach budesonide solutions comprising, as a solubility enhancer, Captisol®. These applications teach sterilization of the budesonide solutions, however the mass loss of budesonide under the reported conditions are considered unacceptable from a commercial standpoint.

There is thus a need for an improved method of terminal sterilization of corticosteroid solutions that results in improved mass loss.

### SUMMARY

The foregoing and other needs are further met by embodiments of the invention, which provide a process of making a sterilized solution of corticosteroid, comprising subjecting a compounded corticosteroid mixture to conditions wherein the mass loss between the starting corticosteroid solution and the sterilized corticosteroid solution is less than about 30%, less than about 25%, less than about 20%, less than about 15%, less than about 10%, less than about 5%, less than about 3%, less than about 2% or about 1% or less. In some embodiments, the corticosteroid solution contains a solubility enhancer, such as a cyclodextrin. In some preferred embodiments, the corticosteroid is budesonide. In some preferred embodiments, the invention provides a process of making a sterilized a solution of corticosteroid, which includes providing a compounded corticosteroid solution and filtering the compounded corticosteroid solution through a filter having a mean pore diameter of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, about 0.15 μm to 0.45 μm, about 0.15 μm to 0.30 μm, about 0.15 μm to 0.25 μm, to produce the sterilized corticosteroid solution. The mass loss due to sterilization procedure is less than about 30%, less than about 25%, less than about 20%, less than about 15%, less than about 10%, less than about 5%, less than about 3%, less than about 2% or about 1% or less of the corticosteroid in the compounded (unsterilized) corticosteroid solution. In some preferred embodiments, the budesonide solution is filtered through a 0.22 μm filter, especially a 0.22 μm PVDF filter, e.g. a Millipore® CVGL71TP3 0.22 μm filter. In some especially preferred embodiments, the mass loss of corticosteroid due to filtering is in the range of about 0.5% to about 30%, about 0.5% to about 25%, about 0.5% to about 20%, about 0.5% to about 15%, about 0.5% to about 10%, about 0.5% to about 5%, about 0.5% to about 2%, about 0.5% to about 1.5% or about 0.5% to about 1.2%.

The foregoing and other needs are further met by embodiments of the invention, which provide a method of reducing the mass loss of corticosteroid in a sterilization process, comprising subjecting a compounded corticosteroid mixture to conditions wherein a mass loss of corticosteroid of less than about 30%, less than about 25%, less than about 20%, less than about 15%, less than about 10%, less than about 5%, less than about 3%, less than about 2% or about 1% or less is achieved. In some embodiments, the corticosteroid solution contains a solubility enhancer, such as a cyclodextrin. In some preferred embodiments, the corticosteroid is budesonide. In some preferred embodiments, the invention provides a method of reducing the mass loss of corticosteroid in a sterilization process, which comprises providing a compounded corticosteroid solution and filtering the compounded corticosteroid solution through a filter having a mean pore size of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm or about 0.15 μm to about 0.45 μm. The mass loss of corticosteroid between the compounded (unfiltered) and sterilized corticosteroid solutions is less than about 30%, less than about 25%, less than about 20%, less than about 15%, less than about 10%, less than about 5%, less than about 3%, less than about 2% or about 1% or less. In some preferred embodiments, the budesonide solution is filtered through a 0.22 μm filter, especially a 0.22 μm PVDF filter, e.g. a Millipore® CVGL71TP3 0.22 μm filter. In some especially preferred embodiments, the mass loss of corticosteroid due to filtering is in the range of about 0.5% to about 30%, about 0.5% to about 25%, about 0.5% to about 20%, about 0.5% to about 15%, about 0.5% to about 10%, about 0.5% to about 5%, about 0.5% to about 2%, about 0.5% to about 1.5% or about 0.5% to about 1.2%.

The foregoing and other needs are further met by embodiments of the invention, which provide a process of making a sterilized mixture of corticosteroid, comprising subjecting a compounded corticosteroid mixture to conditions wherein the concentration of the sterilized corticosteroid mixture is at least about 95%, at least about 96%, at least about 97%, at least about 97.5%, at least about 97.7%, at least about 97.9%, e.g. about 98.2±0.5% or more of the theoretical concentration based upon the starting mass of corticosteroid. In some embodiments, the corticosteroid solution contains a solubility enhancer, such as a cyclodextrin. In some preferred embodiments, the corticosteroid is budesonide. In some preferred embodiments, the invention provides a process of making a sterilized solution of corticosteroid, in which a compounded corticosteroid solution comprising a starting mass of corticosteroid through a filter having a mean pore diameter of about 0.1 μm to about 1.5 μm (e.g. about 0.1, 0.15, 0.2,0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, to produce the sterilized corticosteroid solution. The resulting sterilized corticosteroid solution has a corticosteroid concentration that is at least about 95%, at least about 96%, at least about 97%, at least about 97.5%, at least about 97.7%, at least about 97.9%, e.g. about 98.2±0.5% or more of the theoretical concentration based upon the starting mass of corticosteroid.

The foregoing and other needs are further met by embodiments of the invention, which provide a method of reducing the loss in concentration of corticosteroid in a sterilization process, comprising subjecting a compounded corticosteroid mixture to conditions wherein the concentration of the corticosteroid in the corticosteroid solution has a concentration that is at least about 95%, at least about 96%, at least about 97%, at least about 97.5%, at least about 97.7%, at least about 97.9%, e.g. about 98.2±0.5% or more of the theoretical concentration based on the starting mass of the corticosteroid. In some embodiments, the corticosteroid solution contains a solubility enhancer, such as a cyclodextrin. In some preferred embodiments, the corticosteroid is budesonide. In some preferred embodiments, the invention provides a method of reducing the loss in concentration of corticosteroid in a sterilization process, which process comprises filtering a compounded corticosteroid solution comprising a starting mass of corticosteroid through a filter having a mean pore size of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, to produce a filtered corticosteroid solution. The filtered corticosteroid solution has a concentration that is at least about at least about 95%, at least about 96%, at least about 97%, at least about 97.5%, at least about 97.7%, at least about 97.9%, e.g. about 98.2±0.5% or more of the theoretical concentration based on the starting mass of the corticosteroid.

Other characteristics and advantages of the invention will become apparent to the person of skill in the art upon consideration of the following disclosure.

### BRIEF DESCRIPTION OF THE DRAWINGS

The novel features of the invention are set forth with particularity in the appended claims. A better understanding of the features and advantages of certain embodiments of the present invention will be obtained by reference to the following detailed description that sets forth illustrative embodiments, in which the principles of the invention are utilized, and the accompanying drawings of which:

FIG. 1 is a flow diagram illustrating an embodiment of a budesonide solution manufacturing process according to the present invention.

### INCORPORATION BY REFERENCE

All publications and patent applications mentioned in this specification are herein incorporated by reference to the same extent as if each individual publication or patent application was specifically and individually indicated to be incorporated by reference. In particular, the following WIPO Published Patent Applications, each of which designates the United States, are noted and are specifically incorporated herein in their entireties: WO 2005/065649, WO 2005/065435 and WO 2005/065651.

### DETAILED DESCRIPTION OF THE INVENTION

The present invention relates to the sterilization of corticosteroid mixtures, and in particular budesonide solutions. In particular, the invention provides a method for terminal sterilization of corticosteroid mixtures that is suitable for use in the manufacture of pharmaceutical formulations for use in humans and other mammals. The invention is particularly useful for the sterilization of corticosteroid solutions, especially budesonide solutions. The invention further provides a method of reducing the mass loss of corticosteroids, such as budesonide, during sterilization. The invention further provides a method for reducing the loss in concentration of corticosteroid, such as budesonide, during sterilization. Thus, the invention provides a useful improvement in the manufacture of corticosteroid mixtures, especially corticosteroid solutions, providing a practical method for sterilization without heat, thereby improving the economics of corticosteroid solution manufacture as well as the quality of the final product. Other advantages and characteristics of the present invention will become apparent to the person skilled in the art upon consideration of the following general description and examples.

As used herein, the term “mixture” has its art-recognized meaning in its fullest breadth, including suspensions and solutions. The term “solution” is intended to mean substantially homogeneous mixtures that are substantially clear and free of suspended particulates. The term “compounded mixture” means a mixture in which the pharmaceutical active ingredient has been homogenously mixed with water and is ready to be sterilized. The term “compounded solution” means a solution in which the pharmaceutical active ingredient has been homogeneously dissolved in water and is ready to be sterilized.

In some embodiments, the invention provides a process of making a sterilized solution of corticosteroid, wherein a compounded corticosteroid solution is filtered through a filter having a mean pore diameter of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, about 0.15 μm to about 0.45 μm, about 0.15 μm to about 0.30 μm or about 0.15 μm to about 0.25 μm. Thus, there is produced a sterilized corticosteroid solution. The mass loss between the starting corticosteroid solution and the sterilized corticosteroid solution is less than about 30%, less than about 25%, less than about 20%, less than about 15%, less than about 10%, less than about 5%, less than about 3%, less than about 2% or about 1% or less. In some embodiments, the filter has a mean pore diameter of about 0.2, 0.22 or 0.45 μm. In some embodiments, the filter is a Millipore® CVGL71TP3 0.22 μm filter. In particular embodiments, the corticosteroid is budesonide, although other corticosteroids, and in particular corticosteroids that have low solubility in water, can be used. Particular corticosteroids other than budesonide that may be substituted for budesonide in the process of the present invention are set forth in more detail below. In some embodiments, the filter is a methylcellulose filter or a PVDF filter. Other types of filters are known in the art and may be used. In some preferred embodiments, the filter is a PVDF filter. In some preferred embodiments, the filter is a PVDF filter having a mean pore size of about 0.22 μm, e.g. a Millipore® CVGL71TP3 0.22 μm filter. In particular, it is considered preferable for the corticosteroid solution to include a solubility enhancer. A preferred class of solubility enhancers are the sulfoalkyl ether cyclodextrin derivatives (SAE-CD derivatives), as set forth in WO 2005/065649, WO 2005/065435 and WO 2005/065651. In particular, it is considered advantageous to use a molar excess of solubility enhancer with respect to the corticosteroid. A particularly preferred class of SAE-CD derivatives are the SBE-β-CD compounds, such as SBE7-β-CD (Captisol®), which is available from CyDex, Inc., Lenexa, Kans. Other solubility enhancers that may be included in the solution include Polysorbate 80. Preferred concentrations of Polysorbate 80, when present, include 0.01% and less, 0.005% and less and 0.001% and less; but higher concentrations, e.g. up to 1% and more, may be used. In some preferred embodiments, cyclodextrin and less than about 0.005% (e.g. about 0.001%) Polysorbate 80 are used as solubility enhancers. In particular, compositions comprising an SAE-CD, such as SBE7-β-CD, and excluding Polysorbate 80, are preferred. In some preferred embodiments, the corticosteroid solution also comprises an additional active ingredient, especially a water soluble active ingredient. One class of compounds that is preferably included in the solution are the water soluble short-acting β2-agonists, such as albuterol. In some preferred embodiments, the process results in a mass loss of corticosteroid of less than about 25%, less than about 20%, less than about 15%, less than about 10%, less than about 5%, less than about 3%, less than about 2% or about 1% or less. It is preferred that a filtration step be the sole terminal sterilization step. In some preferred embodiments, however, one or more intermediate filtration steps may be included in the process according to the invention.

In other embodiments, the invention provides a method of reducing the mass loss of corticosteroid in a sterilization process. In some embodiments, the method comprises filtering a starting corticosteroid solution through a filter having a mean pore size of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, about 0.15 μm to about 0.45 μm, about 0.15 μm to about 0.30 μm or about 0.15 μm to about 0.25 μm. A mass loss of corticosteroid of less than about 30%, less than about 25%, less than about 20%, less than about 15%, less than about 10%, less than about 5%, less than about 3%, less than about 2% or about 1% or less is achieved. In some embodiments, the filter has a mean pore diameter of about 0.2, 0.22 or 0.45 μm. In particular embodiments, the corticosteroid is budesonide, although other corticosteroids, and in particular corticosteroids that have low solubility in water, can be used. Particular corticosteroids other than budesonide that may be substituted for budesonide in the process of the present invention are set forth in more detail below. In some embodiments, the filter is a methylcellulose filter or a PVDF filter. Other types of filters are known in the art and may be used. In some preferred embodiments, the filter is a PVDF filter. In some preferred embodiments, the filter is a PVDF filter having a mean pore size of about 0.22 μm, e.g. a Millipore® CVGL7 ITP3 0.22 μm filter. In particular, it is considered preferable for the corticosteroid solution to include a solubility enhancer. A preferred class of solubility enhancers are the sulfoalkyl ether cyclodextrin derivatives (SAE-CD derivatives), as set forth in WO 2005/065649, WO 2005/065435 and WO 2005/065651. In particular, it is considered advantageous to use a molar excess of solubility enhancer with respect to the corticosteroid. A particularly preferred class of SAE-CD derivatives are the SBE-β-CD compounds, such as SBE7-β-CD (Captisol®), which is available from CyDex, Inc., Lenexa, Kans. Other solubility enhancers or compounds that may be included in the solution include Polysorbate 80. Preferred concentrations of Polysorbate 80, when present, include 0.01% and less, 0.005% and less and 0.001% and less. In particular, compositions comprising an SAE-CD, such as SBE7-β-CD, and excluding Polysorbate 80, are preferred. In some preferred embodiments, the corticosteroid solution also comprises an additional active ingredient, especially a water soluble active ingredient. One class of compounds that is preferably included in the solution are the water soluble short-acting 2-agonists, such as albuterol. In some preferred embodiments, the process results in a mass loss of corticosteroid of less than about 30%, less than about 25%, less than about 20%, less than about 15%, less than about 10%, less than about 5%, less than about 3%, less than about 2% or about 1% or less. It is preferred that the filtration step be the sole terminal sterilization step.

In some embodiments, the invention provides a process of making a sterilized solution of corticosteroid, comprising filtering a compounded corticosteroid solution comprising a starting mass of corticosteroid through a filter having a mean pore diameter of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, about 0.15 μm to about 0.45 μm, about 0.15 μm to about 0.30 μm or about 0.15 μm to about 0.25 μm to produce the sterilized corticosteroid solution, whereby the concentration of the sterilized corticosteroid solution is least about 95%, at least about 96%, at least about 97%, at least about 97.5%, at least about 97.7%, at least about 97.9%, e.g. about 98.2±0.5% or more of the theoretical concentration based upon the starting mass of corticosteroid. In some embodiments, the filter has a mean pore diameter of about 0.2, 0.22 or 0.45 μm. In particular embodiments, the corticosteroid is budesonide, although other corticosteroids, and in particular corticosteroids that have low solubility in water, can be used. Particular corticosteroids other than budesonide that may be substituted for budesonide in the process of the present invention are set forth in more detail below. In some embodiments, the filter is a methylcellulose filter or a PVDF filter. Other types of filters are known in the art and may be used. In some preferred embodiments, the filter is a PVDF filter. In some preferred embodiments, the filter is a PVDF filter having a mean pore size of about 0.22 μm, e.g. a Millipore® CVGL71TP3 0.22 μm filter. In particular, it is considered preferable for the corticosteroid solution to include a solubility enhancer. A preferred class of solubility enhancers are the sulfoalkyl ether cyclodextrin derivatives (SAE-CD derivatives), as set forth in WO 2005/065649, WO 2005/065435 and WO 2005/065651. In particular, it is considered advantageous to use a molar excess of solubility enhancer with respect to the corticosteroid. A particularly preferred class of SAE-CD derivatives are the SBE-β-CD compounds, such as SBE7-β-CD (Captisol®), which is available from CyDex, Inc., Lenexa, Kans. Other solubility enhancers that may be included in the solution include Polysorbate 80. Preferred concentrations of Polysorbate 80, when present, include 0.01% and less, 0.005% and less and 0.001% and less. In particular, compositions comprising an SAE-CD, such as SBE7-β-CD, and excluding Polysorbate 80, are preferred. In some preferred embodiments, the corticosteroid solution also comprises an additional active ingredient, especially a water soluble active ingredient. One class of compounds that is preferably included in the solution are the water soluble short-acting β2-agonists, such as albuterol. In some preferred embodiments, the process results in a mass loss of corticosteroid of less than about 30%, less than about 25%, less than about 20%, less than about 15%, less than about 10%, less than about 5%, less than about 3%, less than about 2% or about 1% or less. It is preferred that the filtration step be the sole terminal sterilization step.

The invention further provides a method of reducing the loss in concentration of corticosteroid in a sterilization process, comprising filtering a compounded corticosteroid solution comprising a starting mass of corticosteroid through a filter having a mean pore size of about 0.1 μm to about 1.5 μm (e.g. about 0.1, 0.15, 0.2, 0.22, 0.25, 0.3, 0.35, 0.4, 0.45, 0.5, 0.6, 0.7, 0.8, 0.9, 1.0 μm or up to 1.5 μm), especially about 0.1 μm to 0.5 μm, about 0.15 μm to about 0.45 μm, about 0.15 μm to about 0.30 μm or about 0.15 μm to about 0.25 μm, to produce a filtered corticosteroid solution, wherein the filtered corticosteroid solution has a concentration that is at least about 95%, at least about 96%, at least about 97%, at least about 97.5%, at least about 97.7%, at least about 97.9%, e.g. about 98.2±0.5% or more of the theoretical concentration based on the starting mass of the corticosteroid. In some embodiments, the filter has a mean pore diameter of about 0.2, 0.22 or 0.45 μm. In particular embodiments, the corticosteroid is budesonide, although other corticosteroids, and in particular corticosteroids that have low solubility in water, can be used. Particular corticosteroids other than budesonide that may be substituted for budesonide in the process of the present invention are set forth in more detail below. In some embodiments, the filter is a methylcellulose filter or a PVDF filter. Other types of filters are known in the art and may be used. In some preferred embodiments, the filter is a PVDF filter. In some preferred embodiments, the filter is a PVDF filter having a mean pore size of about 0.22 μm, e.g. a Millipore® CVGL71TP3 0.22 μm filter. In particular, it is considered preferable for the corticosteroid solution to include a solubility enhancer. A preferred class of solubility enhancers are the sulfoalkyl ether cyclodextrin derivatives (SAE-CD derivatives), as set forth in WO 2005/065649, WO 2005/065435 and WO 2005/065651. In particular, it is considered advantageous to use a molar excess of solubility enhancer with respect to the corticosteroid. A particularly preferred class of SAE-CD derivatives are the SBE-β-CD compounds, such as SBE7-β-CD (Captisol®), which is available from CyDex, Inc., Lenexa, Kans. Other solubility enhancers that may be included in the solution include Polysorbate 80. Preferred concentrations of Polysorbate 80, when present, include 0.01% and less, 0.005% and less and 0.001% and less. In particular, compositions comprising an SAE-CD, such as SBE7-β-CD, and excluding Polysorbate 80, are preferred. In some preferred embodiments, the corticosteroid solution also comprises an additional active ingredient, especially a water soluble active ingredient. One class of compounds that is preferably included in the solution are the water soluble short-acting β2-agonists, such as albuterol. In some preferred embodiments, the process results in a mass loss of corticosteroid of less than about 30%, less than about 25%, less than about 20%, less than about 15%, less than about 10%, less than about 5%, less than about 3%, less than about 2% or about 1% or less. It is preferred that the filtration step be the sole terminal sterilization step.

In some embodiments of the invention, the corticosteroid mixture further comprises a solubility enhancer. The term “solubility enhancer” means a pharmaceutically inert ingredient that enhances the solubility of corticosteroid in water or that enhances the ability of the corticosteroid to form a clear mixture that is substantially free of suspended particles. In some embodiments, the solubility enhancer can have a concentration (w/v) ranging from about 0.0001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 5% to about 10% when the solubility enhancer is a cyclodextrin or cyclodextrin derivative.

A “solubility enhancer,” as used herein, includes one or more compounds which increase the solubility of corticosteroid in the aqueous phase of the corticosteroid mixture. In general the solubility enhancer increases the solubility of the corticosteroid in water without chemically changing the corticosteroid. In particular, the solubility enhancer increases the solubility of corticosteroid without substantially decreasing, and in some embodiments increasing, the activity of the corticosteroid.

Solubility enhancers are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Solubility enhancers suitable for use in the present invention include, but are not limited to, propylene glycol, non-ionic surfactants, phospholipids, cyclodextrins and derivatives thereof, and surface modifiers and/or stabilizers.

Examples of non-ionic surfactants which appear to have a particularly good physiological compatibility for use in the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the trade name Tweens 20-40-60, etc.), Polysorbate 80, Polyethylene glycol 400; sodium lauryl sulfate; sorbitan laurate, sorbitan palmitate, sorbitan stearate (available under the trade name Span 20-40-60 etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the present invention are formulated with the corticosteroid to form liposome preparations, micelles or mixed micelles. Methods for the preparations and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vemuri et al. (Preparation and characterization of liposomes as therapeutic delivery systems: a review in Pharm Acta Helv. 1995, 70(2):95-111) and U.S. Pat. Nos. 5,019,394, 5,192,228, 5,882,679, 6,656,497 each of which is specifically incorporated by reference herein.

In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. In the context of the present invention, both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solubilized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 nm to about 100 nm. Particularly preferred are micelles with average diameters of about 10 to about 50 nm. Methods of producing micelles and mixed micelles are known in the art and described in, for example, U.S. Pat. Nos. 5,747,066 and 6,906,042, each of which is specifically incorporated by reference herein.

Phospholipids are amphiphilic lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, tryglycerides and other substances.

Additional phospholipids which are suitable according to the present invention on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phopholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the corticosteroid to form colloidal structures. Colloidal solutions are mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319, which is specifically incorporated by reference herein.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, β-cyclodextrins, γ-cyclodextrins, SAE-CD derivatives (e.g., SBE-α-CD, SBE-β-CD (Captisol°), and SBE-γ-CD) (CyDex, Inc. Lenexa, Kans.), hydroxyethyl, hydroxypropyl (including 2-and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β- and γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999).

Suitable surface modifiers for use in the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the present invention include, but are not limited to, hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens , e.g., Tween 20™ and Tween 80™ (ICI Specialty Chemicals)), polyethylene glycols (e.g., Carbowaxs 3550™ and 934™ (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68™ and F108™, which are block copolymers of ethylene oxide and propylene oxide), poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)), Tetronic 1508™ (T-1508) (BASF Wyandotte Corporation), Tritons X-200™, which is an alkyl aryl polyether sulfonate (Rohm and Haas), Crodestas F-100™, which is a mixture of sucrose stearate and sucrose distearate (Croda Inc.), p-isononylphenoxypoly-(glycidol), also known as Olin-10G™ or Surfactant 10™ (Olin Chemicals, Stamford, Conn.), Crodestas SL-40.RTM. (Croda, Inc.), and SA9OHCO, which is C₁₈H₃₇CH₂(—CON(CH₃)—CH₂(CHOH)₄(CH₂OH)₂ (Eastman Kodak Co.), decanoyl-N-methylglucamide, n-decyl-β-D-glucopyranoside, n-decyl-β-D-maltopyranoside, n-dodecyl β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonanoyl-β-D-glucopyranoside, octanoyl-N-methylglucamide, n-octyl-β-D-glucopyranoside, octyl β-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like. (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Other useful cationic stabilizers include, but are not limited to, cationic lipids, sulfonium, phosphonium, and quarternary anmonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅ dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈) dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, Mirapol™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides, imide azolinium salts, protonated quaternary acrylamides, methylated quaternary polymers, such as poly[diallyl dimethylammonium chloride] and poly-[N-methyl vinyl pyridinium chloride], and cationic guar.

In addition to aqueous mixtures comprising a corticosteroid and a solubility enhancer, it is contemplated herein that aqueous inhalation mixtures formulated by methods which provide enhanced solubility are likewise suitable for use in the presently disclosed invention. Thus, in the context of the present invention, a “solubility enhancer” includes aqueous inhalation mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Specific solubility enhancers that may be mentioned within the scope of the invention include polysorbate 80 and SAE-CD derivatives, SBE-α-CD, SBE-β-CD, SBE-β-CD and dimethyl β-CD, hydroxypropyl-β-cyclodextrin, 2-HP-β-CD. In particular embodiments, SAE-CD derivatives are preferred. In particularly preferred embodiments, the SAE-CD derivatives belonging to the group of SBE-β-CD derivatives are preferred. In specific embodiments, a particularly preferred solubility enhancer is SBE7-β-CD. In some embodiments, Polysorbate 80 is included in the formulation at concentrations of about 0.01% or less, especially about 0.005% or less, and more specifically about 0.001% or less; while in other embodiments it is preferred to substantially exclude Polysorbate 80 from the corticosteroid solution. In some preferred embodiments, the corticosteroid solution contains a molar excess of SAE-CD derivative, especially SBE7-β-CD, with respect to the corticosteroid, especially budesonide.

The term corticosteroid is intended to have the full breadth understood by those of skill in the art. Particular corticosteroids contemplated within the scope of the invention are those that are not generally soluble in water to a degree suitable for pharmaceutical administration, and thus require the presence of a solubility enhancer to dissolve them in aqueous solution. Particular corticosteroids that may be mentioned in this regard include those set forth in WO 2005/065649, WO 2005/065435 and WO 2005/065651. See in particular page 46 of WO 2005/065651, which is incorporated hereinby reference. The corticosteroids that may be substituted for budesonide include aldosterone, beclomethasone, betamethasone, ciclesonide, cloprednol, cortisone, cortivazol, deoxycortone, desonide, desoximetasone, dexamethasone, difluorocortolone, fluclorolone, flumethasone, flunisolide, flucinolone, fluocinonide, fluocortin butyl, fluocortisone, flurocortolone, fluorometholone, flurandrenolone, fluticasone, halcinonide, hydrocortisone, icomethasone, meprednisone, methylpredinsolone, mometasone, paramethasone, prednisolone, prednisone, rofleponide, RPR 106541, tixocortol, triamcinolone and their pharmaceutically active derivatives, including prodrugs and pharmaceutically acceptable salts. In some embodiments, two or more corticosteroids from the foregoing list may be combined in a solution according to the present invention. In some embodiments, budesonide may be combined with one or more of the corticosteroids from the foregoing list.

The concentration of corticosteroid in the corticosteroid composition may vary from about 1 μg/ml to about 2000 μg/ml, about 1 μg/ml to about 1000 μg/ml or about 1 to about 500 μg/ml, especially about 50 μg/ml to about 500 μg/ml, or about 100 to about 400 μg/ml. Particular values that may be mentioned are about 1, about 5 μg/ml, about 10 μg/ml, about 20 μg/ml, about 50 μg/ml, about 100 μg/ml and about 200 μg/ml and about 250 μg/ml. In some preferred embodiments, the corticosteroid concentration in the sterilized solution is in the range of about 80 μg/ml to about 480 μg/ml, especially about 80 μg/ml, about 120 μg/ml, about 240 μg/ml or about 480 μg/ml.

In addition to corticosteroid, the corticosteroid solution may include other active ingredients, especially other water-soluble active ingredients. Particularly suitable active ingredients are those that act either in conjunction with, or synergistically with, the corticosteroid for the treatment of one or more respiratory disorders (such as asthma or chronic obstructive pulmonary disease (CODP)) or symptoms of respiratory disease, such as bronchial spasm, inflammation of bronchia, increased phlegm viscosity, decreased lung capacity, etc. The corticosteroid thus may be compounded with one or more other drugs, such as β₂ adrenoreceptor agonists (such as albuterol), dopamine D₂ receptor antagonists, anticholinergic agents or topical anesthetics. Specific active ingredients are known in the art, and preferred embodiments are set forth on pages 48-49 of WO 2005/065651, which pages are expressly incorporated herein by reference in their entirety.

In some embodiments, other active ingredients, especially water soluble active ingredients are included in the corticosteroid solution. In some preferred embodiments, the corticosteroid solution includes a water soluble short acting 2-agonist, such as albuterol. Thus, some preferred embodiments include budesonide, a molar excess (relative to budesonide) of a cyclodextrin solubility enhancer, such as SBE7-β-CD, and albuterol.

In some preferred embodiments, the corticosteroid solution is manufactured by mixing a mass of corticosteroid starting material with the other ingredients in a high sheer mixer for less than about 5, less than about 4, less than about 3 and in particular about 2 hours or less. Preferably, such mixing is conducted under an oxygen-depleted atmosphere, such as under nitrogen or argon gas positive pressure, particularly under nitrogen gas. In particular embodiments, the mixing is carried out in a high sheer mixer having a capacity of at least about 10 L, at least about 50 L, at least about 100 L, at least about 250 L or at least about 500 L. In some such preferred embodiments, the mixing is carried out with alternating cycles of vacuum and overlay with positive inert gas (such as N₂ or Ar) pressure. In some specific embodiments, after mixing the solution is stored under an inert gas overlay (N₂ or Ar) of at least about 50 mbar, at least about 100 mbar, at least about 200 mbar, at least about 500 mbar or about 1200 mbar or more. The mixing, the storage or both are performed under an N₂ overlay of about 1200 mbar. (All pressures are gauge pressures unless otherwise indicated).

Although the invention has been described with reference to filtration as the sole sterilization step, the person skilled in the art will recognize that filtration may be combined with other sterilization techniques, such as heat treatment and/or irradiation. It is also possible to perform either heat treatment, irradiation or both on a compounded corticosteroid mixture, although terminal filtration is preferred.

As used herein, the term “mass loss” refers to the difference in mass of budesonide in the sterilized budesonide solution as compared to the mass of budesonide in the starting budesonide solution. The mass loss is conveniently measured in terms of percent mass loss according to the following formula:

% mass loss=100%*(M₁−M₂)/M₁,

where M₁ is the mass of budesonide of the starting budesonide solution and M₂ is the mass of budesonide in the sterilized budesonide solution.

The percent concentration decrease can be computed in a like manner. Thus the formula for % concentration decrease is:

% concentration decrease=100%*(C₁−C₂)/C₁,

where C₁ is the concentration of the corticosteroid in solution prior to filtration, C₂ is the concentration of the corticosteroid in solution after filtration. The concentration values C₁ and C₂ may be expressed in any suitable units, such as molarity (mole per liter), molality (moles per kg), grams of solute per liter of solution or grams of solute per kg of solution, so long as they are both expressed in the same units. Where the concentration C₁ is not assayed prior to filtration, it may be calculated based upon the amount (mass) of corticosteroid starting material added to the mixing apparatus and the mass or volume of the resulting solution.

Corticosteroid solutions prepared by methods according to the invention are used to treat one or more respiratory disorders. The corticosteroid solutions are advantageously compounded such that the active pharmaceutical ingredients contained therein are available on a unit dosage basis in a therapeutically effective amount. A therapeutically effective amount or effective amount is that amount of a pharmaceutical agent to achieve a pharmacological effect. The term “therapeutically effective amount” includes, for example, a prophylactically effective amount. An “effective amount” of a corticosteroid, such as budesonide, is an amount effective to achieve a desired pharmacologic effect or therapeutic improvement without undue adverse side effects. The effective amount of a corticosteroid, such as budesonide, will be selected by those skilled in the art depending on the particular patient and the disease level. It is understood that “an effective amount” or “a therapeutically effective amount” can vary from subject to subject, due to variation in metabolism of a corticosteroid, such as budesonide, age, weight, general condition of the subject, the condition being treated, the severity of the condition being treated, and the judgment of the prescribing physician.

The terms “treat” and “treatment” as used in the context of a bronchoconstrictive disorder refer to any treatment of a disorder or disease related to the contraction of the bronchia, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

Specific disorders that may be treated with compositions of the invention include, but are not limited to, respiratory diseases characterized by bronchial spasm, bronchial inflammation, increased phlegm viscosity, decreased lung capacity, etc. Specific conditions that may be treated include asthma, reactive airway disease and chronic obstructive pulmonary disease (COPD).

Manufacturing Corticosteroid Solutions

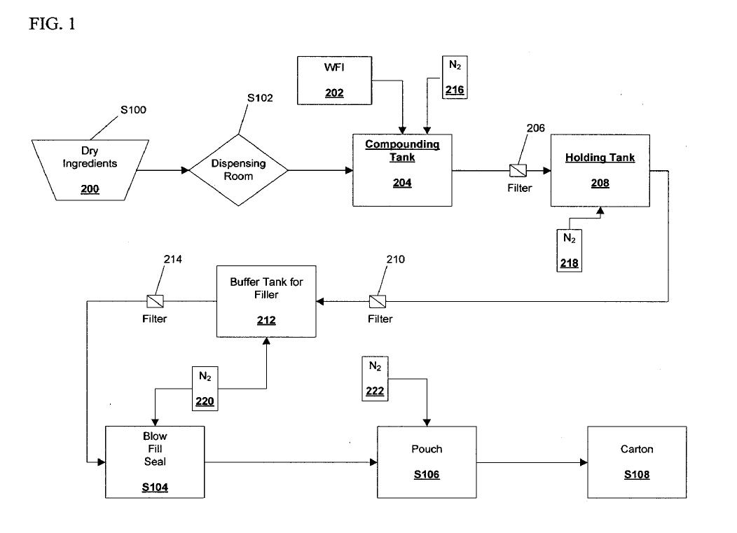

A process according to the present invention is illustrated in FIG. 1. In S100, dry ingredients 200 are identified and are assayed to determine their water content. Dry ingredients 200 include corticosteroid (e.g. budesonide, and particularly micronized budesonide) and cyclodextrin (e.g. Captisol® cyclodextrin), as well as additional ingredients, such as citric acid, sodium citrate, sodium chloride and sodium EDTA (sodium edetate). In S102, the ingredients 200 are moved to a dispensing room and are weighed and placed in containers suitable for dispensing the ingredients into the compounding tank 204. The cyclodextrin is advantageously divided into three aliquots; and the corticosteroid (e.g. budesonide) is placed in a suitable container. Water for injection (WFI) 202 is charged into the compounding tank 204. The dry ingredients 200 are then added to the compounding tank 204. At least a portion of the mixing in the compounding tank 204 is conducted under oxygen-depleted conditions. For example, the WFI 202 may have been sparged with nitrogen or argon to remove dissolved oxygen. Alternatively, the compounding tank 204 may be sealed and subjected to one or more (preferably two) cycles of vacuum/hold/overpressure with inert gas 216 (such as nitrogen or argon) during the mixing process. The overpressure of inert gas 216 may be a value above atmospheric pressure (any positive gauge pressure), and may for example be in the range of from 100 mbar to about 3000 mbar. In currently preferred embodiments, the overpressure is about 1,200 mbar of nitrogen gas. In some embodiments, the compounding tank 204 is fitted with a homogenization apparatus that is designed to create high shear conditions. In some embodiments, the compounding tank 204 is a FrymaKoruma Dinex® (FrymaKoruma GmbH, Neuenburg, Germany) compounding mixer, which comprises a holding tank with a water jacket, an inlet for introducing liquid ingredients (e.g. WFI), a homogenizer, a stirrer, a short loop, a long loop and a funnel for introducing dry ingredients. High shear conditions in the FrymaKoruma Dinex® compounding mixer are approximately 1000 rpm to 4000 rpm, preferably about 1500 rpm to about 3000 rpm. For the 500 L batch size in a compounding tank 204 designed to accommodate a maximum volume of 500 L, one preferred homogenizer speed is about 2,500 rpm, although other values may be selected by one having skill in the art. For a 50 L batch size in a compounding tank 204 designed to accommodate a maximum volume of 500 L, one preferred homogenizer speed is about 1,700 rpm, although other values may be selected by one having skill in the art. The compounding tank 204 may be sealed to exclude atmospheric gasses. The compounding tank 204 may be any suitable size, in particular about 50L to 1000L capacity. The 500L model is currently preferred. At the end of mixing (e.g. 30 to 600 min, and preferably about 120 min.) the corticosteroid (e.g. budesonide) solution is discharged under pressure into a holding tank 208. In some embodiments, a filter 206 is located between the compounding tank 204 and the holding tank 208. The filter may be a 0.1 to 0.22 μm mean pore diameter filter (preferably a 0.22 μm mean pore diameter) of a suitable composition (e.g. PVDF), e.g. a Millipore® CVGL71TP3 0.22 μm filter.

The corticosteroid (e.g. budesonide) solution may be held in the holding tank 208 for a period of time, e.g. up to seven days. The holding tank 208 may be air-tight and may be charged with an overpressure of inert gas 218, such as nitrogen or argon. In general, the inert gas pressure should be held well above atmospheric pressure, e.g. about 2000 mbar. The corticosteroid (e.g. budesonide) solution is next discharged under pressure into a buffer tank 212. The buffer tank 212 provides a mechanical buffer between the holding tank 208 and the filler in the Blow Fill Seal step S104. The buffer tank may also have a inert gas 220 overlay. A filter 210 may be interposed between the holding tank 208 and the buffer tank 212. When present, the filter 210 may be a 0.1 to 0.22 μm mean pore diameter filter (preferably a 0.22 μm mean pore diameter) of a suitable composition (e.g. PVDF), e.g. a Millipore® CVGL71TP3 0.22 μm filter.

The budesonide solution is discharged from the buffer tank 212 to a Blow Fill Seal apparatus in step S104. A filter 214 may be interposed between the buffer tank 212 and the Blow Fill Seal apparatus in step S104. When present, the filter 214 may be a 0.1 to 0.22 μm filter (preferably a 0.22 μm PVDF filter), e.g. a Millipore® CVGL71TP3 0.22 μm filter. The Blow Fill Seal step S104 entails dispensing the liquid corticosteroid (e.g. budesonide) solution into individual pharmaceutically acceptable containers (referred to elsewhere herein as bottles, ampoules or vials) and sealing the individual containers. In some embodiments, the containers are LDPE ampoules having a nominal capacity of 0.5 ml, although other materials and sizes are within the skill in the art. In some embodiments, the Blow Fill Seal step S104 may be conducted under oxygen-depleted conditions, such as positive inert gas 220 (e.g. nitrogen) pressure. The individual containers are then packaged in pouches in the Pouch step S106. In some embodiments, the Pouch step S106 may be carried out under oxygen-depleted conditions, such as under positive inert gas 222 (e.g. nitrogen) pressure. Each pouch may contain one or more containers (e.g. ampoules or vials) of corticosteroid (e.g. budesonide). In some embodiments, each pouch contains 1, 2, 3, 4, 5, 6, 7, 8, 9, 10, 11, 12, 13, 14 or more containers. In some currently preferred embodiments, each pouch contains 5 ampoules. The pouches are packaged into cartons in the Carton step S108.

As used herein, sterility is determined by an art-recognized method, e.g. by the USP <71>, PhEur 2.6.1 or other art-recognized method of measuring sterility.

### EXAMPLES

### Example 1

### Preparation of 120 Microgram/Milliliter Budesonide Solution

A 50 L batch of budesonide solution (nominally 120 μg/ml) was prepared according to the following procedure:

Prior to weighing the Captisole cyclodextrin (Cyclodextrin) and budesonide, the starting materials were assayed. The assay values were used to calculate the actual amount of Cyclodextrin and budesonide starting materials to be used in the formulation. The Cyclodextrin was found to be 4.9% water (95.1% Cyclodextrin). Thus, the total amount of Cyclodextrin starting material was increased by a proportional amount. It was calculated that the amount of Cyclodextrin starting material needed was 935.8569 g (representing 890.0 g Cyclodextrin). This Cyclodextrin starting material was weighed out in three measure: 735.86 g, 100.0 g and 100.0 g. In the same way, the budesonide starting material was assayed and found to contain 98.2% budesonide base. The amount of budesonide starting material was then calculated to be 5.95 μ/0.982=6.06 g. Thus, 6.06 g of budesonide starting material was weighed out.

The following additional ingredients were weighed out: 15.0 g citric acid anhydrous; 25.0 g sodium citrate dihydrate USP. Sufficient water for injection to make up 50 kg of solution was also provided.

The mixing apparatus comprised a high sheer mixer a feed funnel in an isolator, as well as a vacuum apparatus and a source of nitrogen gas. The high sheer mixer was enclosed, thereby making it possible to apply a vacuum to the contents of the mixer during mixing.

Precisely 40 kg of water were introduced into to a mixing apparatus (FrymaKouma Dinex® 700 vacuum processor, 500 L max volume). A 224 mbar vacuum was taken on the mixing apparatus and held for 5 minutes. Then 1278 mbar (gauge pressure) of nitrogen gas was introduced into the mixing vessel, which remained isolated from atmosphere outside the mixer during the duration of the mixing procedure. About one third of the Captisol® cyclodextrin (Cyclodextrin) was added to the funnel in the isolator. Then about 100.0 g of Cyclodextrin was added to the budesonide starting material in an Erlenmeyer flask and shaken until a homogeneous mixture was formed. This mixture was then added to the feed funnel. Then 100.0 g of Cyclodextrin was added to the Erlemneyer flask and shaken until homogeneous. The contents of the Erlenmeyer flask were then added to the funnel. Finally 15.0 g citric acid anhydrous, 25.0 sodium citrate dihydrate USP, 5.0 g sodium EDTA dihydrate and 325.0 g sodium chloride were each sequentially added to the funnel. When all the ingredients had been combined in the funnel, all were introduced to the mixer by vacuum suction.

The contents of the mixer were then homogenized at 1500 rpm for about 5 minutes at about 17° C. The Erlenmeyer flask that formerly contained the budesonide starting material was then rinsed twice with about 150 ml water; and the rinse water was added to the funnel. Abut half of the remaining water was added to the funnel and the contents of the funnel were introduced into the mixer by vacuum suction. Then the final quantity of water was added to the funnel and introduced into the mixer by vacuum suction. Finally, the homogenizer speed was increased to 1700 rpm for 120 minutes.

During the 120 minute homogenization, the mixing tank was purged of oxygen as follows: (1) A first vacuum of about 200 mbar was applied and held for about 5 minutes; (2) a nitrogen pressure of 1200 mbar was applied; (3) a second vacuum of about 200 mbar was applied and held for about 5 minutes; and (4) a second nitrogen overlay of about 1215 mbar was applied to the mixer. At the end of homogenization, samples of the homogenized budesonide solution were taken and sent to Q.C.

### Example 2

### Sterilization Procedure

The homogenized budesonide solution from Example 1 was filtered through a 0.22 μm Millipore (CVGL71TP3) filter through a Teflon® hose into a sterilized holding tank. An overpressure of about 1200 mbar of nitrogen was applied to the filtered solution.

After the sterilized budesonide solution was collected in the holding tank, it was assayed. The budesonide solution was found to contain 98.2±0.5% of the theoretical concentration of budesonide, based upon the amount of budesonide in the budesonide starting material. The solution passed sterility according to USP <71> and PhEur 2.6.1.

As can be seen from Example 2, the present invention provides a method of sterilizing a budesonide solution, wherein the mass loss and the decrease in budesonide concentration levels is low. The invention this provides a practical method for making sterilized budesonide solutions that are suitable for inhalation therapy.

### Example 3

### 80 Microgram/Milliliter Budesonide Solution (Batch G1059)

A 50 L batch of budesonide solution having a final concentration of approximately 80 μg/ml was prepared according to the following procedure.

First budesonide and Captisol® cyclodextrin (Cyclodextrin) were assayed to determine the percent water in each sample. The target mass of cyclodextrin in the 50 L batch was 595 g; and the target mass of budesonide was 4.1 g. The assay for Cyclodextrin gave a value of 4.8% water or 95.2% Cyclodextrin; the budesonide assay gave a percent budesonide value of 99.2%. Thus, the amount of Cyclodextrin was calculated to be 595 g/0.952=625 g Cyclodextrin; the budesonide mass was calculated to be 4.1 g/0.992=4.133 g budesonide.

The cyclodextrin was weighed out in three aliquots of 100 g, 100 g and 425 g of cyclodextrin, respectively. Precisely 4.133 g of budesonide were weighed out in a container (budesonide container).

A cleaned holding tank was steam sterilized and 40 kg of water for injection (WFI) were charged into the holding tank. A clean stainless steel 500 L (max capacity) FrymaKoruma Dinex® mixing vessel (mixing tank) with a stirrer and homogenizer was steam sterilized for 10 minutes and dried. The mixing tank is equipped with a short homogenization loop (short loop) and a funnel for introduction of dry ingredients (dry-addition funnel; funnel). The 40 kg of water were then transferred to the mixing tank from the holding tank under pressure. Approximately half of the pre-weighed 425 g aliquot of Cyclodextrin were then added to the dry-addition funnel. The entire contents of the budesonide container were then added to the funnel, taking care not to allow any of the budesonide to contact the walls of the funnel. The first 100 g aliquot of Cyclodextrin was then added to the budesonide container and shaken to scavenge any residual budesonide. The contents of the budesonide container were then added to the funnel. This procedure was repeated with the second 100 g aliquot of Cyclodextrin.

The following quantities of ingredients were then added to the funnel: 15.0 of anhydrous citric acid, 25.0 g of sodium citrate dihydrate, 5.0 g sodium edetate dihydrate, 395.0 g of sodium chloride and the second half of Cyclodextrin from the 425 g aliquot. With the stirrer set to 25 rpm and the homogenizer set to 1500 rpm, the entire contents of the dry funnel were added to the mixing tank under suction. The contents of the mixing tank were then homogenized through the short loop for approximately 10 minutes.

The budesonide container was then washed with two 150 g aliquots of WFI: A first 150 g aliquot of WFI was added to the budesonide container and shaken. The contents of the budesonide container were then added to the funnel. This procedure was repeated with a second 150 g aliquot of WFI and then the entire contents (˜300 ml) of the funnel were added to the mixing tank by suction. Approximately half of 8.631 kg of WFI was added to the funnel. The WFI in the funnel was then added to the mixing tank by suction. This procedure was repeated with the remaining approximately half of the 8.631 kg of WFI.

The homogenizer speed was increased to 1700 rpm. The mixing tank was then purged with nitrogen (N₂): A vacuum of −200 mbar was applied to the mixing tank and held for five minutes; then the mixing tank was pressurized with 1,200 mbar of nitrogen. This procedure was repeated once. Samples of budesonide solution were drawn from the mixing tank through a 0.22 μm PVDF filter at 60, 90 and 120 minutes. At the end of 124 minutes, the entire contents of the mixing tank were discharged through Teflon® PTFE hose and a 0.22 μm Durapore® PVDF cartridge filter and into a holding tank. The procedure netted 46.6 kg of 80.2 μg/ml (assay value) budesonide solution. The budesonide solution was blow filled into LDPE vials to produce filled vials containing 0.53 ml/vial (42.1 μg/vial of budesonide). The solution passed sterility according to USP <71> and PhEur 2.6.1.

### Example 4

### 40, 60, 120 and 240 μg/0.5 mL Dose Budesonide Solutions

Following the general procedures outlined in Examples 1-3, above, budesonide solutions having concentrations of 80, 120, 240 and 480 μg/mL were prepared, dispensed into LDPE vials (ampoules) in 0.5 mL doses and pouched as described above. The resulting 0.5 mL doses contained 40, 60, 120 and 240 μg budesonide per 0.5 mL dose. The amounts of each ingredient contained in each ampoule are set forth in Table 1, below. The solutions passed sterility according to USP <71> and PhEur 2.6.1.

TABLE 1
40, 60, 120 and 240 μg/0.5 mL Dose Budesonide

| | 240 mcg/ | 120 mcg/ | 60 mcg/ | 40 mcg/ |
|---|---|---|---|---|
| Ingredient | 0.5 mL | 0.5 mL | 0.5 mL | 0.5 mL |
| Budesonide | 0.048 | 0.024 | 0.012 | 0.008 |
| Captisol | 7.5 | 3.57 | 1.78 | 1.19 |
| Citric acid | 0.03 | 0.03 | 0.03 | 0.03 |
| Sodium Citrate | 0.05 | 0.05 | 0.05 | 0.05 |
| Dihydrate USP | | | | |
| NaCl | 0.45 | 0.57 | 0.73 | 0.79 |
| Na-EDTA | 0.01 | 0.01 | 0.01 | 0.01 |
| Water | ad 100.0 | ad 100.0 | ad 100.0 | ad 100.0 |

Values shown are [w %]; Osmolality adjusted to 290 mOsm/kg; pH 4.5

### Example 5

### Mass Loss Across Multiple Batches

Following the general manufacturing procedures outlined in Examples 1-4, above, the batches set forth in Table 2 were prepared. The nominal concentration of each batch (approximating 80 μg/mL, 120 μg/mL, 240 μg/L or 480 μg/L) is shown in the column marked “Nominal μg/mL”. An in-process test was performed, wherein budesonide solution was extracted from the solution through a 0.22 μm PVDF syringe filter after dissolution. The IPC budesonide concentration data are given in the column labeled “IPC μg/mL.” The column marked Δ% IPC shows the difference between the nominal concentration and the IPC filtered solution. At the end of processing, the final budesonide solution (“Release”) was assayed and the concentration of budesonide was determined in the “Release” solution. These data are summarized for each batch in the column labeled “Release μg/mL.” The percent difference between the “Release” concentration of budesonide and the nominal concentration is set forth for each batch in the column labeled “Δ% Release.” Each solution passed sterility according to USP <71> and PhEur 2.6.1.

TABLE 2
Dissolution Data for Multiple Batches

| | | | | | | | Batch |
|---|---|---|---|---|---|---|---|
| | Nominal | IPC | % Δ | | Release | Batch No. | size |
| PARI Batch Code | μg/mL | μg/mL | IPC | % Δ Release | μg/mL | Holopack | [kg] |
| HP001 MED120_0 | 240 | 235.1 | −2.04 | −3.96 | 230.5 | FI141 | 50 |
| HP002 MED120_1 | 240 | 232.5 | −3.13 | −2.13 | 234.9 | FJ032A | 50 |
| HP005 LOW60_1 | 120 | 124.01 | 3.34 | 1.08 | 121.3 | FJ037 | 50 |
| HP007 HIGH240_1 | 480 | 494.3 | 2.98 | −0.31 | 478.5 | FJ097 | 50 |
| HP008 LOW60_2 | 120 | 124.0 | 3.33 | −1.92 | 117.7 | FJ102 | 50 |
| HP010 MED120-EDTA | 240 | 242.9 | 1.21 | −0.08 | 239.8 | FJ111 | 50 |
| HP011 MED120_2 | 240 | 241.9 | 0.79 | 0.46 | 241.1 | FJ114 | 50 |
| HP012 LOW60_3 | 120 | 120.7 | 0.58 | −0.58 | 119.3 | FJ110 | 50 |
| HP013 MED120+PS80 | 240 | 242.5 | 1.04 | −0.79 | 238.1 | FJ115 | 50 |
| HP014 HIGH240_2 | 480 | 483.1 | 0.65 | 0.48 | 482.3 | FJ113 | 50 |
| HP016 MED120_3 | 240 | 233.9 | −2.54 | −3.75 | 231 | GB098 | 500 |
| HP018 LOW60_4 | 120 | 118.3 | −1.42 | −3.92 | 115.3 | GB111 | 500 |
| HP020 LOW60_5 | 120 | 116.0 | −3.33 | −7.33 | 111.2 | GB131 | 50 |
| HP021 MED120_4 | 240 | 231 | −3.75 | −7.21 | 222.7 | GD060 | 50 |
| HP023 LOW60_6 | 120 | 114.98 | −4.18 | −7.54 | 110.95 | GD064 | 50 |
| HP025 LOW40_1 | 80 | 78.13 | −2.34 | −7.10 | 74.32 | GD083 | 50 |
| HP026 LOW60_7 | 124.8 | 113.3 | −9.21 | −8.89 | 113.7 | GE090 | 50 |
| HP027 MED120_5 | 249.6 | 233.9 | −6.29 | −6.29 | 233.9 | GE099 (A) | 500 |
| HP029 LOW60_8 | 124.8 | 112.2 | −10.10 | −11.62 | 110.3 | GE129 | 50 |
| HP030 LOW60_9 | 124.8 | 112.2 | −10.10 | −5.93 | 117.4 | GE150 | 50 |
| HP031 LOW60_10 | 124.8 | 112.4 | −9.94 | −8.73 | 113.9 | GE166 | 50 |
| HP032 MED120_6 | 240 | 234 | −2.5 | −2.92 | 233 | GG202 | 50 |
| HP033 LOW60_11 | 124.8 | 123.1 | −1.36 | −2.48 | 121.7 | GG207 | 50 |
| HP034 MED120_7 | 249.6 | 247.1 | −1.00 | −1.00 | 247.1 | GG213 | 50 |
| HP035 LOW60_12 | 122.3 | 120.8 | −1.23 | −0.49 | 121.7 | GI047 | 500 |
| HP037 LOW40_2 | 81.4 | 79.9 | −1.84 | −1.47 | 80.2 | GI059 | 50 |
| HP038 LOW60_13 | 122.3 | 119.9 | −1.96 | −1.39 | 120.6 | GI070 | 50 |
| HP039 MED120_8 | 245.3 | 239.2 | −2.49 | −1.71 | 241.1 | GI079 | 50 |

Although preferred embodiments of the present invention have been shown and described herein, it will be apparent to those skilled in the art that such embodiments are provided by way of example only. Numerous variations, changes, and substitutions will be apparent to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered herein.

What is claimed is:

1. A process of making a sterilized solution of corticosteroid, comprising filtering a compounded corticosteroid solution through a filter to produce the sterilized corticosteroid solution, wherein the mass loss between the starting corticosteroid solution and the sterilized corticosteroid solution is less than about 30%.

2. The process of claim 1, wherein the filter has a mean pore diameter of about 0.1 μm to about 1.5 μm.

3. The process of claim 2, wherein the filter has a mean pore diameter of about 0.1 μm to about 0.5 μm.

4. The process according to claim 3, wherein the filter has a mean pore diameter of about 0.22 μm.

5. The process of claim 1, wherein the corticosteroid is budesonide.

6. The process of claim 1, wherein the filter is a methylcellulose filter or a PVDF filter.

7. The process of claim 6, wherein the filter is a PVDF filter having a mean pore diameter of about 0.22 μm.

8. The process of claim 1, wherein the starting corticosteroid solution further comprises a solubility enhancer.

9. The process of claim 8, wherein the starting corticosteroid solution comprises a molar excess of a solubility enhancer as compared to corticosteroid.

10. The process of claim 9, wherein the solubility enhancer is selected from the group consisting of sulfoalkyl ether cyclodextrins (SAE-CDs).

11. The process of claim 10, wherein the solubility enhancer is the sulfoalkyl ether cyclodextrin SBE7-β-CD (Captisol®).

12. The process of claim 1, wherein the corticosteroid solution further comprises albuterol.

13. The process of claim 1, wherein the solubility enhancer comprises cyclodextrin and about 0.001% Polysorbate 80.

14. The process of claim 1, wherein the sterilized corticosteroid solution has a mass loss of less than about 10%.

15. The process of claim 14, wherein the sterilized corticosteroid solution has a mass loss of less than about 5%.

16. The process of claim 15, wherein the sterilized corticosteroid solution has a mass loss of less than about 2%.

17. A method of reducing the mass loss of corticosteroid in a sterilization process, comprising filtering a starting corticosteroid solution through a filter to produce a sterilized corticosteroid solution, wherein a mass loss of corticosteroid of less than about 30% is achieved.

18. The method of claim 17, wherein the filter has a mean pore diameter of about 0.1 μm to about 1.5 μm.

19. The method of claim 18, wherein the filter has a mean pore diameter of about 0.1 μm to about 0.5 μm.

20. The method according to claim 19, wherein the filter has a mean pore diameter of about 0.22 μm.

21. The method of claim 17, wherein the corticosteroid is budesonide.

22. The method of claim 17, wherein the filter is a methylcellulose filter or a PVDF filter.

23. The method of claim 22, wherein the filter is a PVDF filter having a mean pore diameter of about 0.22 μm.

24. The method of claim 17, wherein the starting corticosteroid solution further comprises a solubility enhancer.

25. The method of claim 24, wherein the starting corticosteroid solution comprises a molar excess of a solubility enhancer as compared to corticosteroid.

26. The method of claim 25, wherein the solubility enhancer is selected from the group consisting of sulfoalkyl ether cyclodextrins (SAE-CDs).

27. The method of claim 26, wherein the solubility enhancer is the sulfoalkyl ether cyclodextrin SBE7-β-CD (Captisol®).

28. The method of claim 17, wherein the corticosteroid solution further comprises albuterol.

29. The method of claim 17, wherein the solubility enhancer comprises cyclodextrin and about 0.001% Polysorbate 80.

30. The method of claim 17, wherein the sterilized corticosteroid solution has a mass loss of less than about 10%.

31. The method of claim 30, wherein the sterilized corticosteroid solution has a mass loss of less than about 5%.

32. The method of claim 31, wherein the sterilized corticosteroid solution has a mass loss of less than about 2%.

33. A process of making a sterilized solution of corticosteroid, comprising filtering a compounded corticosteroid solution comprising a starting mass of corticosteroid through a filter to produce the sterilized corticosteroid solution, whereby the concentration of the sterilized corticosteroid solution is at least about least about 95%, at least about 96%, at least about 97%, at least about 97.5%, at least about 97.7%, at least about 97.9%, e.g. about 98.2±0.5% or more of the concentration of corticosteroid in the compounded corticosteroid solution.

34. The process of claim 33, wherein the filter has a mean pore diameter of about 0.1 μm to about 1.5 μm.

35. The process of claim 34, wherein the filter has a mean pore diameter of about 0.1 μm to about 0.5 μm.

36. The process according to claim 35, wherein the filter has a mean pore diameter of about 0.22 μm.

37. The process of claim 33, wherein the corticosteroid is budesonide.

38. The process of claim 33, wherein the filter is a methylcellulose filter or a PVDF filter.

39. The process of claim 38, wherein the filter is a PVDF filter having a mean pore diameter of about 0.22 μm.

40. The process of claim 33, wherein the starting corticosteroid solution further comprises a solubility enhancer.

41. The process of claim 40, wherein the starting corticosteroid solution comprises a molar excess of a solubility enhancer as compared to corticosteroid.

42. The process of claim 40, wherein the solubility enhancer is selected from the group consisting of sulfoalkyl ether cyclodextrins (SAE-CDs).

43. The process of claim 42, wherein the solubility enhancer is the sulfoalkyl ether cyclodextrin SBE7-β-CD (Captisol®).

44. The process of claim 33, wherein the corticosteroid solution further comprises albuterol.

45. The process of claim 33, wherein the solubility enhancer comprises cyclodextrin and about 0.001% Polysorbate 80.

46. The process of one of claim 33, wherein the sterilized corticosteroid solution has a concentration that is at least about 95% of the theoretical concentration based upon the starting mass of corticosteroid.

47. The process of claim 46, wherein the sterilized corticosteroid solution has a concentration that is at least about 96% of the theoretical concentration based upon the starting mass of corticosteroid.

48. The process of claim 47, wherein the sterilized corticosteroid solution has a concentration that is at least about 97% of the theoretical concentration based upon the starting mass of corticosteroid.

49. The process of claim 48, wherein the sterilized corticosteroid solution has a concentration that is at least about 97.5% of the theoretical concentration based upon the starting mass of corticosteroid.

50. The process of claim 49, wherein the sterilized corticosteroid solution has a concentration that is at least about 97.7% of the theoretical concentration based upon the starting mass of corticosteroid.

51. A method of reducing the loss in concentration of corticosteroid in a sterilization process, comprising filtering a compounded corticosteroid solution comprising a starting mass of corticosteroid through a filter to produce a filtered corticosteroid solution, wherein the filtered corticosteroid solution has a concentration that is at least about 90.0% of the theoretical concentration based on the starting mass of the corticosteroid.

52. The method of claim 51, wherein the filter has a mean pore diameter of about 0.1 μm to 0.5 μm.

53. The method according to claim 51, wherein the filter has a mean pore diameter of about 0.22 μm.

54. The method of claim 51, wherein the corticosteroid is budesonide.

55. The method of claim 51, wherein the filter is a methylcellulose filter or a PVDF filter.

56. The method of claim 55, wherein the filter is a PVDF filter having a mean pore diameter of about 0.22 μm.

57. The method of claim 51, wherein the starting corticosteroid solution further comprises a solubility enhancer.

58. The method of claim 57, wherein the starting corticosteroid solution comprises a molar excess of a solubility enhancer as compared to corticosteroid.

59. The method of claim 58, wherein the solubility enhancer is selected from the group consisting of sulfoalkyl ether cyclodextrins (SAE-CDs).

60. The method of claim 59, wherein the solubility enhancer is the sulfoalkyl ether cyclodextrin SBE7-β-CD (Captisol®).

61. The method of claim 51, wherein the corticosteroid solution finther comprises albuterol.

62. The method of claim 51, wherein the solubility enhancer comprises cyclodextrin and about 0.001% Polysorbate 80.

63. The method claim 51, wherein the sterilized corticosteroid solution has a concentration that is at least about 95% of the theoretical concentration based upon the starting mass of corticosteroid.

64. The method of claim 63, wherein the sterilized corticosteroid solution has a concentration that is at least about 96% of the theoretical concentration based upon the starting mass of corticosteroid.

65. The method of claim 64, wherein the sterilized corticosteroid solution has a concentration that is at least about 97% of the theoretical concentration based upon the starting mass of corticosteroid.

66. The method of claim 65, wherein the sterilized corticosteroid solution has a concentration that is at least about 97.5% of the theoretical concentration based upon the starting mass of corticosteroid.

67. The method claim 66, wherein the sterilized corticosteroid solution has a concentration that is at least about 97.7% of the theoretical concentration based upon the starting mass of corticosteroid.

68. The method claim 67, wherein the sterilized corticosteroid solution has a concentration that is at least about 97.9% of the theoretical concentration based upon the starting mass of corticosteroid.

69. The method of claim 68, wherein the sterilized corticosteroid solution has concentration that is at least about 98.2±0.5% of the theoretical concentration based upon the starting mass of corticosteroid.

70. A process of making a sterilized solution of corticosteroid, comprising subjecting a compounded corticosteroid solution to conditions wherein the mass loss between the starting corticosteroid solution and the sterilized corticosteroid solution is less than about 30%, less than about 25%, less than about 20%, less than about 15%, less than about 10%, less than about 5%, less than about 3%, less than about 2% or about 1% or less.

71. A method of reducing the nass loss of corticosteroid in a sterilization process, comprising subjecting a compounded corticosteroid solution to conditions wherein a mass loss of corticosteroid of less than about 30%, less than about 25%, less than about 20%, less than about 15%, less than about 10%, less than about 5%, less than about 3%, less than about 2% or about 1% or less is achieved.

72. A process of making a sterilized solution of corticosteroid, comprising subjecting a compounded corticosteroid solution to conditions wherein the concentration of the sterilized corticosteroid solution is at least about 95%, at least about 96%, at least about 97%, at least about 97.5%, at least about 97.7%, at least about 97.9%, e.g. about 98.2±0.5% or more or more of the theoretical concentration based upon the starting mass of corticosteroid.

73. A method of reducing the loss in concentration of corticosteroid in a sterilization process, comprising subjecting a compounded corticosteroid solution to conditions wherein the concentration of the corticosteroid in the corticosteroid solution has a concentration that is at least about 95%, at least about 96%, at least about 97%, at least about 97.5%, at least about 97.7%, at least about 97.9%, e.g. about 98.2±0.5% or more or more of the theoretical concentration based upon based on the starting mass of the corticosteroid.
